(19) United States
(12) Patent Application Publication
(10) Pub. No.: US 2014/0288579 A1
(43) Pub. Date: Sep. 25, 2014

(54) Ultrasonic Follicle Unit Extraction Device And Method

(71) Applicant: Sanusi Umar, Redondo Beach, CA (US)

(72) Inventor: Sanusi Umar, Redondo Beach, CA (US)

(21) Appl. No.: 14/090,968

(22) Filed: Nov. 26, 2013

Related U.S. Application Data
(60) Provisional application No. 61/729,733, filed on Nov. 26, 2012.

(51) Int. Cl. A45D 26/00; A61B 17/32

(52) U.S. Cl. CPC A45D 26/0057; A61B 17/320068
USPC 606/133

(57) ABSTRACT

A follicle unit extraction device comprises a generally tubular body having a distal cutting end region, and an ultrasonic transducer coupled to the punch to apply rapid, incremental, distally-directed cutting force pulses via the punch to the tissue at a donor site to thereby offer a more precisely controllable methodology for penetrating the tissue while the punch is positioned at the donor site.

A generally tubular follicle punch having a distal cutting end region is brought into contact with the skin at the donor site. The punch is inserted distally into the skin in such a way that the follicle enters the punch's interior as the skin and underlying tissue circumscribing the follicle are cut, and ultrasonic, incremental, distally-directed cutting force pulses are applied to the tissue via the punch during the cutting process.

### FIELD OF THE INVENTION

This invention relates to follicle unit extraction devices and methods.

### BACKGROUND OF THE INVENTION

Hair transplantation is a surgical technique that involves moving skin containing hair follicles from one part of the body (the donor site) to bald or balding parts (the recipient site).

Hair naturally grows in follicles that contain groupings of 1 to 4 hairs, and transplant techniques typically move the 1-4 hair “follicular units” from the donor site to the recipient site.

The follicles of hair are typically removed from the donor site using punches of between 0.7 mm and 1.25 mm in diameter. The punches are tubular bodies having a skin-contacting cutting edge, and are typically mounted in a tool that causes the punch to rotate as the punch is brought into contact with the donor site. Hair follicles are very easily damage during the removal process, and damaged follicles are unlikely to be successfully transplanted.

### SUMMARY OF THE INVENTION

A follicle unit extraction device comprises a generally tubular follicle punch body having a distal cutting end region. In practice, the punch is positioned at a donor site during the extraction process, and may be rotated slightly, so that tissue surrounding the follicle is cut without damaging contact with the follicle. An ultrasonic transducer is coupled to the punch to impart a vibratory cutting force against the tissue.

The foregoing process may be performed manually or under machine or computer control. In addition, a mechanism for automatically rotating the punch may be employed, and may accordingly be coupled to the ultrasonic transducer.

These and further details of the invention will be apparent to those of ordinary skill in the art from reading a description of the currently preferred embodiment of the invention described below.

### DETAILED DESCRIPTION OF THE PREFERRED EMBODIMENT

A follicle punch comprises a generally tubular body extending from a proximal end to a skin-contacting distal end about a generally central axis. The distal end terminates in a cutting edge that is preferably on the punch's outer diameter

The punch is inserted into the skin at a donor site in such a way that the hair enters the punch's interior. The punch is advanced past the follicle. Once the punch has penetrated sufficiently, the hair follicle is removed from the donor site for subsequent transfer to a recipient site.

To penetrate the skin, the punch can be manually pressed distally by hand. An ultrasonic transducer is coupled to the punch to apply rapid, incremental, distally-directed cutting force pulses to the tissue via the punch to thereby offer a more precisely controllable methodology for penetrating the tissue while the punch is positioned at the donor site.

Although the present invention and its advantages have been described in detail, it should be understood that various changes, substitutions and alterations can be made herein without departing from the spirit and scope of the invention as will be defined by appended claims.

I claim:

1. A method for cutting a follicle unit at a donor site prior to extraction comprising the step of applying ultrasonic vibratory cutting force against the tissue at the donor site during the cutting operation;

2. A method for cutting a follicle unit at a donor site prior to extraction comprising the steps of:
bringing a generally tubular follicle punch having a distal cutting end region into contact with the skin at a donor site;
inserting the punch distally into the skin in such a way that the follicle enters the punch's interior as the skin and underlying tissue circumscribing the follicle are cut; and
applying ultrasonic, incremental, distally-directed cutting force pulses to the tissue via the punch during the cutting process.

3. A follicle unit cutting system comprising
a generally tubular follicle punch having a distal cutting edge, and
an ultrasonic transducer coupled to the punch to impart rapid, incremental, distally-directed cutting force pulses to the punch during the cutting operation.
